(19) United States
(12) Patent Application Publication
(10) Pub. No.: US 2007/0249732 A1
(43) Pub. Date: Oct. 25, 2007

(54) USE OF CERTAIN PHENYL NAPTHYL COMPOUNDS FOR PROTECTION OF NEURONS AND OLIGODENDROCYTES IN THE TREATMENT OF MULTIPLE SCLEROSIS (MS)

(75) Inventors: Jean MERRILL, Whippany, NJ (US); Sandrine FUNES, Nogent-sur-marne (FR); Wayne PETKO, South Bound Brook, NJ (US); Frederike WIRTZ-BRUGGER, Branchburg, NJ (US); Karen CHANDROSS, Somerset, NJ (US)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: AVENTIS PHARMACEUTICALS INC., Bridgewater, NJ (US)

(21) Appl. No.: 11/757,604

(22) Filed: Jun. 4, 2007

Related U.S. Application Data
(63) Continuation of application No. PCT/US05/45490, filed on Dec. 14, 2005.
(60) Provisional application No. 60/641,315, filed on Jan. 4, 2005.

(51) Int. Cl. A61K 31/05; A61P 25/00

(52) U.S. Cl. 514/732

(57) ABSTRACT

Certain biphenyl compounds, including 5-chloro-6-(4-hydroxyphenyl)-2-naphthalenol and 6-(4-hydroxy-phenyl)-2-naphthalene methanol are useful in the treatment of multiple sclerosis on the cellular level of the central nervous system through the protection of the patients' oligodendrocytes and neurons.

### CROSS-REFERENCE TO RELATED APPLICATIONS

This application is a continuation of International Patent Application No. PCT/US2005/045490 filed on Dec. 14, 2005 which is incorporated herein by reference in its entirety which also claims the benefit of priority of U.S. Provisional Patent Application No.60/641,315 filed on Jan. 4, 2005.

### FIELD OF THE INVENTION

The present invention relates to methods of treating multiple sclerosis. In particular, the present invention relates to the protection of neurons and/or oligodendrocytes in multiple sclerosis patients with compounds of formula I, as well as their isomers, racemates, enantiomers, their salts, and medicaments containing them.

### BACKGROUND OF THE INVENTION

Multiple sclerosis (MS) is an autoimmune disease that leads to a loss of CNS (central nervous system) myelin, oligodendrocyte cell death and axonal destruction, causing severe functional deficits. MS occurs at a 2-3 times higher incidence in women than men (Duquette, et al., 1992. Can. J. Neurol. Sci. 19: 466-71.) and estrogen reduces disease severity during the second and third trimesters of pregnancy (Confavreux et al., 1998. N Eng J Med 339: 285-291), whereas the clinical symptoms of MS have been reported to exacerbate after delivery (Evron et al., 1984. Am. J. Reprod. Immunol. 5:109-113; Mertin and Rumjanek 1985. J. Neurol Sci. 68:15-24; Grossman, 1989. J. Steroid Biochem. 34: 241-245; Confavreux et al., 1998. N. Engl. J. Med. 339: 285-291). Treatment with estriol decreases gadolinium enhancing lesions and MRI volume (Voskuhl and Palaszynski, 2001. Neuroscientist. 7(3): 258-270; Sicotte et al., 2002. Ann Neurol. 52: 421-428). Furthermore, estrogens cause immune response shifts, amelioration of clinical symptoms and enhanced myelin formation in rodent EAE (experimental allergic encephalomyelitis) (Curry and Heim 1966. Nature 81: 1263-1272; Kim et al., 1999. Neurology. 52: 1230-1238; Ito et al., 2002. Clin Immunol. 102(3): 275-282). Estrogen has been reported to protect oligodendrocytes from cytotoxicity induced cell death (Takao et al., 2004. J Neurochem. 89: 660-673) and 17β-estradiol (E2) has been reported to hasten the elaboration of multiple, interconnecting processes on oligodendrocytes (Zhang et al., 2004. J Neurochem 89: 674-684).

There is increasing evidence that estrogen plays a direct protective role in response to degenerative disease and injury by enhancing cell survival, axonal sprouting, regenerative responses, synaptic transmission, and neurogenesis. In the CNS, there is increased synthesis of estrogen and enhanced expression of the estrogen receptors at sites of injury (Garcia-Segura et al., 2001. Prog. in Neurobiol. 63: 29-60.) and estrogen-mediated cellular protection has been demonstrated in a number of in vitro models of neurodegeneration, including β-amyloid induced cytotoxic, excitotoxicity, and oxidative stress (BehI et al., 1995. Biochem. Biophys. Res. Commun. 216,473-482; Goodman et al., 1996. J. Neurochem. 66:1836-1844; Green et al., 1997. J. Neurosci. 17: 511-515; Behl et al., 1999. Trends Pharmacol. Sci. 20: 441-444). Recent clinical studies suggest that estrogen replacement therapy may also decrease the risk and delay the onset and progression of Alzheimer's disease and schizophrenia. (For a review see Garcia-Segura et al., 2001. Prog. in Neurobiol. 63: 29-60.) E2, a lipophilic hormone that can cross the blood-brain barrier, maintains brain systems sub-serving arousal, attention, mood, and cognition (Lee and McEwan, 2001. Annu. Rev. Pharmacol. & Toxicol. 41: 569-591.). In addition, both natural estrogens and synthetic selective estrogen receptor modulators (SERMs), such as tamoxifen, decrease neuronal damage caused by ischemic stroke, whilst either E2 or raloxifene protect neurons against 1-methly-4-phenyl-1,2,3,6 tetrahydropyridine-induced toxicity (Callier, et al., 2001. Synapse 41: 131-138; Dhandapani and Brann, 2003. Endocrine 21: 59-66).

Estrogen's neuroprotective effects are mediated through the modulation of bcl-2 expression, activation of cAMP and mitogen-activated kinase signaling pathways, modulation of intracellular calcium homeostasis, enhancement of antioxidant activity, and/or activation of estrogen receptors (ER) that can act as hormone-regulated transcription factors (Mangelsdorf, et al., 1995. Cell 83: 835-839; Katzenellenbogen, et al., 1996. Mol. Endocrinol. 10: 119-131; Singer et al., 1996. Neurosci. Left. 212: 13-16; Singer et al., 1998. Neuroreport 9: 2565-2568; Singer et al., 1999. Neurosci. Left. 212: 13-16; Weaver et al., 1997. . Brain Res. 761: 338-341; Watters and Dorsa, 1998. J. Neurosci. 18: 6672-6680; Singh et al., 1999. J. Neurosci. 19: 1179-1188; Alkayed et al., 2001. J. Neurosci. 21: 7543-7550; Garcia-Segura et al., 2001. Prog. in Neurobiol. 63: 29-60). Two characterized estrogen receptors, ERα and ERβ, belong to the class I hormone receptor family that functions as nuclear transcription factors. ERα and ERβ (in the form of mRNA or protein) are expressed in neural cell types including Schwann cells, the myelin forming cells of the peripheral nervous system, and CNS neurons, astrocytes and oligodendrocytes (Miranda and Toran-Allerand, 1992; Santagati, et al., 1994; Kuiper, et al., 1996; Mosselman, et al., 1996; Thi et al. 1998; Platania, et al., 2003). In oligodendrocytes, the myelin forming cells of the CNS that are lost in MS, ERα has been reported to be nuclear, whereas ERβ is cytolpasmic, in vivo immunoreactivity being readily detectable in cytoplasm and myelin sheaths (Zhang et al., 2004. J Neurochem 89: 674-684). Recently Arvanitis at al., 2004 (J Neurosci Res. 75: 603-613) have reported an ER with similarities to ERβ in isolated CNS myelin, the myelin sheath of spinal cord and brain sections and the oligodendrocyte plasma membrane.

Mimicking and/or enhancing the beneficial effects of estrogen in MS by means of small molecules that are ligands at ERβ, or compounds that preferentially mimic the effects of estrogen at sites other than the classical ERα is likely to have advantages for the treatment of MS in that the small molecules would be devoid of the untoward “hormonal” effects of estrogen which are mediated by ERα. These other ER sites may include the recently identified ER-X, which has been identified in neurons and is developmentally regulated (Toran-Allerand 2004. Endocrinology 145:1069-1074), or GPR30, which allows estrogen to trigger different pathways that integrate cell surface signaling with gene transcription (Kanda and Watanabe 2003. J Invest Derm 121: 771-780).

These compounds may also be used to treat or prevent the development of other demyelinating diseases, including Charcot-Marie-Tooth disease, Pelizaeus-Merzbacher disease, encephalomyelitis, neuromyelitis optica, adrenoleukodystrophy, Guillian-Barre syndrome, and disorders in which myelin-forming glial cells (oligodendrocytes or Schwann cells) are damaged, including spinal cord injury, neuropathies and nerve injury.

### SUMMARY OF THE INVENTION

A subject of the invention is a new use of certain biphenyl compounds for the treatment of multiple sclerosis. The compounds of the present invention are those comprising:
5-chloro-6-(4-hydroxyphenyl)-2-naphthalenol 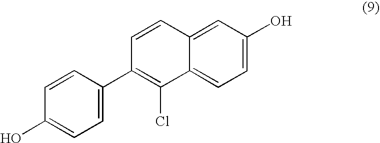
and ps 6-(4-hydroxy-phenyl)-2-Naphthalenemethanol 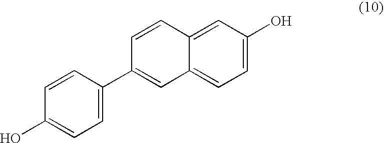
are useful for providing protection to oligodendrocytes and neurons of multiple sclerosis patients.

### DETAILED DESCRIPTION OF THE INVENTION

The invention also relates to the addition salts of the foregoing compounds with inorganic or organic acids.

Compounds which contain one or more asymmetric centers have isomeric forms; these isomers and mixtures form part of the invention. The racemates and the enantiomers of these compounds also form part of the invention. The above compounds, which contain one or more asymmetric centers, have isomeric forms; these isomers and mixtures form part of the invention. The racemates and the enantiomers of these compounds also form part of the invention.

The compounds used in the process of this invention can be prepared by synthetic processes known in the art, in particular, those disclosed in U.S. Pat. No. 6,147,119 to LeSuisse et al. which is hereby incorporated by reference.

Terms Used Herein have the Meanings Defined in this Specification.

a) “Pharmaceutically acceptable salts” means either an acid addition salt or a basic addition salt, whichever is possible to make with the compounds of the present invention.

“Pharmaceutically acceptable acid addition salt” is any non-toxic organic or inorganic acid addition salt of the base compounds represented by Formula I. Illustrative inorganic acids which form suitable salts include hydrochloric, hydrobromic, sulfuric and phosphoric acid and acid metal salts such as sodium monohydrogen orthophosphate and potassium hydrogen sulfate. Illustrative organic acids which form suitable salts include the mono-, di- and tri-carboxylic acids. Illustrative of such acids are, for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicyclic, 2-phenoxybenzoic, p-toluenesulfonic acid and sulfonic acids such as methanesulfonic acid and 2-hydroxyethanesulfonic acid. Either the mono- or di-acid salts can be formed, and such salts can exist in either a hydrated or substantially anhydrous form. In general, the acid addition salts of these compounds are more soluble in water and various hydrophilic organic solvents and which in comparison to their free base forms, generally demonstrate higher melting points.

“Pharmaceutically acceptable basic addition salts” means non-toxic organic or inorganic basic addition salts of the compounds of Formula 1. Examples are alkali metal or alkaline-earth metal hydroxides such as sodium, potassium, calcium, magnesium or barium hydroxides; ammonia, and aliphatic, alicyclic, or aromatic organic amines such as methylamine, trimethylamine and picoline. The selection of the appropriate salt may be important so that the ester is not hydrolyzed. The selection criteria for the appropriate salt will be known to one skilled in the art.

“Patient” means a warm blooded animal, such as for example rat, mice, dogs, cats, guinea pigs, and primates such as humans.

b) “Treat” or “treating” means any treatment, including, but not limited to, alleviating symptoms, eliminating the causation of the symptoms either on a temporary or permanent basis, or preventing or slowing the appearance of symptoms and progression of the named disorder or condition.

c) “Therapeutically effective amount” means an amount of the compound, which is effective in treating the named disorder or condition.

d) “Pharmaceutically acceptable carrier” is a non-toxic solvent, dispersant, excipient, adjuvant or other material which is mixed with the compound of the present invention in order to permit the formation of a pharmaceutical composition, i.e., a dosage form capable of administration to the patient. One example of such a carrier is pharmaceutically acceptable oil typically used for parenteral administration.

e) “Stereoisomers” is a general term for all isomers of the individual molecules that differ only in the orientation of their atoms in space. It includes mirror image isomers (enantiomers), geometric (cis/trans) isomers, and isomers of compounds with more than one chiral center that are not mirror images of one another (diastereoisomers).

In treating a patient afflicted with a condition described above, a compound of Formula (I) can be administered in any form or mode which makes the compound bioavailable in therapeutically effective amounts, including orally, sublingually, buccally, subcutaneously, intramuscularly, intravenously, transdermally, intranasally, rectally, topically, and the like. One skilled in the art of preparing formulations can determine the proper form and mode of administration depending upon the particular characteristics of the compound selected for the condition or disease to be treated, the stage of the disease, the condition of the patient and other relevant circumstances. For example, see Remington's Pharmaceutical Sciences, 18^{th} Edition, Mack Publishing Co. (1990), incorporated herein by reference.

The compositions of the present invention may be administered orally, for example, in the form of tablets, troches, capsules, elixirs, suspensions, solutions, syrups, wafers, chewing gums and the like and may contain one or more of the following adjuvants: binders such as microcrystalline cellulose, gum tragacanth or gelatin; excipients such as starch or lactose, disintegrating agents such as alginic acid, Primogel, corn starch and the like; lubricants such as magnesium stearate or Sterotex; glidants such as colloidal silicon dioxide; and sweetening agents such as sucrose or saccharin may be added or a flavoring agent such as peppermint, methyl salicylate or orange flavoring. When the dosage unit form is a capsule, it may contain, in addition to materials of the above type, a liquid carrier such as polyethylene glycol or a fatty oil. Other dosage unit forms may contain other various materials, which modify the physical form of the dosage unit, for example, as coatings. Thus, tablets or pills may be coated with sugar, shellac, or other enteric coating agents. A syrup may contain, in addition to the present compounds, sucrose as a sweetening agent and certain preservatives, dyes and colorings and flavors.

The solutions or suspensions may also include one or more of the following adjuvants, sterile diluents such as water for injection, saline solution, fixed oils, polyethylene glycols, glycerin, propylene glycol or other synthetic solvents; antibacterial agents such as benzyl alcohol or methyl paraben; antioxidants such as ascorbic acid or sodium bisulfite; chelating agents such as ethylene diaminetetraacetic acid; buffers such as acetates, citrates or phosphates and agents for the adjustment of tonicity such as sodium chloride or dextrose. The parenteral preparation can be enclosed in ampules, disposable syringes or multiple dose vials.

The dosage range at which compounds of Formula I exhibit their ability to act therapeutically can vary depending upon the particular compound, the severity of the condition, the patient, the formulation, other underlying disease states that the patient is suffering from, and other medications that may be concurrently administered to the patient. Generally, the compounds of Formula I will exhibit their therapeutic activities at dosages of between about 0.001 mg/kg of patient body weight/day to about 100 mg/kg of patient body weight/day.

The contents of all publications and patents discussed herein are hereby incorporated herein by reference. It will be appreciated that every suitable combination of the respective elements of the present invention may be interchanged with one or more of other similar, suitable components known in the art and changed in minor, non-functional respects. These additional embodiments of the invention are also regarded as falling within the scope of the claims herein.

The examples detailed below are provided to better describe and more specifically set forth the elements and mechanics/operation of the present invention as contemplated by the inventors herein, but for obvious reasons the examples cannot describe all of them. It is to be recognized that the examples therefore are for illustrative purposes only however, and should not be interpreted as limiting the spirit and scope of the invention as later recited by the claims that follow.

### EXAMPLES

### I. Binding Assay

A number of compounds were tested for ERα and ERβ binding affinity using Panvera's Fluorescence Polarization Competition Assay Kits (cat. nos. P2698 and P2700). Briefly, ERα or ERβ were thawed on ice from −80° C. An estrogen receptor and fluorescent ligand (Fluormone TM) complex was formed at a 15/1 molar ratio for ERα and at a 10/1 molar ratio for ERβ (2X complex). Serial dilutions of the test compounds were made in assay buffer and the assay was initiated by adding 50 ul of the 2X receptor-ligand complex to 50 ul of compound solution in black 96-well plates. Zero percent competition (theoretical maximum polarization) was measured in wells containing 50 ul of buffer and 50 ul of the 2X receptor-ligand complex. The plates were incubated after gentle shaking in the dark at room temperature. Polarization values (mP) were read no longer than 7 hours after the reaction was started with a FARCyte Fluorescent reader (Amersham) at excitation and emission wavelengths of 485 nm and 535 nm, respectively. Data was analyzed using non-linear regression and IC50 values determined using GraphPad Prism. Estradiol was used as the reference compound.

### II. Oligodendrocyte Toxicity Assay

Primary rat oligodendrocyte progenitor cells were obtained from the cerebra of 2-3 day old postnatal rats (Sprague Dawley). The meninges were removed and tissue was mechanically dissociated. Cells were plated on T75 flasks and fed with DMEM +10% FBS.

Enriched OLPs were collected by mechanical separation from the astrocytic monolayer and were expanded in serum free media (SFM) supplemented with the mitogens, PDGF-AA (10 ng/mI) and FGF-2 (10 ng/mI).

To generate mature oligodendrocytes, progenitor cells were switched to SFM supplemented with IGF-1 (10ng/ml) 24 hours after plating and cells were grown under these conditions for 7 days prior to experimental assays.

Cells were plated in 96-well plates, 10,000 per well. Medium was changed to fresh medium and cells were pretreated with compounds for 1 hour. Toxins were added to give the following final concentrations: Sin-110 mM, Camptothecin 1 μM

After 24 hours, medium was removed and assayed for LDH activity using the Promega cytotox 96 kit (catalog# G1780). Results were calculated as percent protection against toxin-induced toxicity.

These compounds have been assessed for their efficacy in protection against cell death produced by toxic agents such as SIN-1 (3-morpholino-sydnonimine, producing peroxynitrite) and camptothecin. The target cells assessed in vitro are primary cultures of rodent oligodendrocyte progenitors and their mature counterparts.

| | | Progenitor | | | | |
|---|---|---|---|---|---|---|
| | | Oligodendrocytes | | Mature | Affinity | |
| | | | Campto- | Oligodendrocytes | for ER | |
| | | SIN-1 | thecin | SIN-1 | α | β |
| Compound | Structure | IC50 | IC50 | IC50 | nM | nM |
| 6-(4-hydroxy-phenyl)- 2- Naphthalenemethanol | 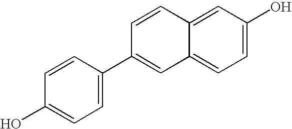 | 0.167 | 2.18 | 18 | 416 | 28 |
| 5-chloro-6-(4- hydroxyphenyl)-2- naphthalenol | 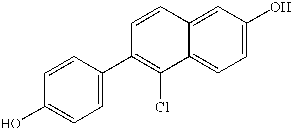 | 2.169 | <3 | >30 | 35.4 | 3 |

What is claimed is:

1. A method for the treatment of multiple sclerosis to a patient having the disease by protecting the central nervous system neurons or oligodendrocytes which comprises the administration of a therapeutically effective amount of a compound selected from the group consisting of 5-chloro-6-(4-hydroxyphenyl)-2-naphthalenol and 6-(4-hydroxy-phenyl)-2-naphthalene methanol, its isomers, racemates and enantiomers, a non-toxic organic or inorganic acid addition salt thereof or an inorganic or organic moiety that may be identical or different and consist of a hydrogen atom, or an alkyl group containing from 1 to 4 organic acids.

2. The method of claim 1 wherein said effective amount is administered daily and is in the range from about 0.001 to about 100 mg/kg of patient body wt./day.

3. The method of claim 2 wherein said effective amount of the compound comprises a pharmaceutical composition which is suitable for administration to the patient orally, sublingually, buccally, subcutaneously, intramuscularly, intravenously, transdermally, intranasally, rectally, and topically.
